(19) United States
(12) Patent Application Publication
(10) Pub. No.: US 2024/0398857 A1
(43) Pub. Date: Dec. 5, 2024

(54) COMPOSITIONS FOR MANAGEMENT OF HPV (HUMAN PAPILLOMA VIRUS) INFECTIONS

(71) Applicant: PAPILLEX INC., Oakville (CA)

(72) Inventor: Elizabeth GOLDSPINK, Oakville (CA)

(73) Assignee: PAPILLEX INC., Oakville (CA)

(21) Appl. No.: 18/677,013

(22) Filed: May 29, 2024

Related U.S. Application Data
(60) Provisional application No. 63/504,856, filed on May 30, 2023.

(51) Int. Cl. A61K 33/30; A61K 31/07; A61K 31/353; A61K 31/375; A61K 31/519; A61K 31/714; A61K 33/04; A61K 36/074; A61K 36/31; A61K 36/481; A61K 36/82; A61K 45/06; A61P 31/20

(52) U.S. Cl. CPC A61K 33/30; A61K 31/07; A61K 31/353; A61K 31/375; A61K 31/519; A61K 31/714; A61K 33/04; A61K 36/071; A61K 36/074; A61K 36/31; A61K 36/481; A61K 36/82; A61K 45/06; A61P 31/20

(57) ABSTRACT

A composition for the management of Human Papillomavirus (HPV) infections is provided. The composition comprises a combination of activated folate, one or more zinc compounds such as zinc sulfate, a phenol catechin such as Epigallocatechin-3-gallate (EGCG), broccoli seed or sprout, and one or more carotenoids. The composition may comprise one or more mushrooms or mushroom extracts. The composition may be useful for the control of symptoms associated with HPV infection in one or more individuals, and may also include reducing the frequency and/or persistence of HPV related conditions.

### CROSS-REFERENCE TO RELATED APPLICATION

The present application claims priority to U.S. Provisional Patent Application Ser. No. 63/504,856, the entire contents of which are hereby incorporated by reference.

### FIELD

The present application relates to a novel composition for the management of Human Papillomavirus (HPV) infections. In certain embodiments, the composition comprises a mixture of active ingredients that may inhibit or reduce HPV viral replication likely through various mechanisms, thus may provide a comprehensive approach to managing such a viral infection.

### BACKGROUND

Human Papillomavirus (HPV) refers to a group of more than 200 related viruses, which are among the most common sexually transmitted infections worldwide. Certain types of HPV, particularly types 16 and 18, are high-risk and can lead to various cancers, including cervical, anal, oropharyngeal, and others. Other types, notably 6 and 11, can cause warts in both the genital and respiratory areas. Most HPV infections are asymptomatic and clear on their own. However, persistent infections by high-risk types can lead to cellular changes and, over time, can cause cancer. HPV is so prevalent that nearly all sexually active men and women are likely to get at least one type of HPV at some point in their lives.

### HPV Symptoms

An HPV outbreak (an “outbreak”) typically refers to the manifestation of symptoms caused by the Human Papillomavirus (HPV). While most HPV infections are asymptomatic and clear up on their own, some can lead to skin or mucosal changes. These can appear as warts or lesions in the anogenital region mouth, or throat. Genital warts are the most common symptom of an HPV outbreak. They may appear as small, flesh-coloured or gray swellings in the genital area, sometimes clustering to form a cauliflower-like shape. In other cases, persistent infection with high-risk HPV types can lead to precancerous changes, particularly in the cervix, which may not present immediate symptoms but can be detected through regular screenings. An HPV outbreak's severity, appearance, and location can vary depending on the specific strain(s) of HPV involved.

### HPV Frequency

HPV outbreaks and frequency thereof, specifically referring to the appearance of symptoms such as warts, can vary greatly in nature and severity due to multiple factors, such as the specific HPV strain(s), the individual's immune response, and potential unknown triggers. Some people may experience frequent outbreaks, while others may never have noticeable symptoms despite being infected. The triggers for an outbreak are not fully understood but may include factors like a weakened immune system, stress, other infections, or hormonal changes. The timing and frequency of outbreaks can also be unpredictable. Some people may experience several outbreaks in a year, while others may have an initial outbreak and never have another one, or the symptoms may appear years after the initial infection. It underscores the unpredictable and highly individual nature of an HPV infection.

While there is no known cure at present, it is considered generally useful and desirable to reduce both the severity of HPV symptoms and frequency of HPV outbreaks.

This background information is provided for making information available believed by the applicant to be of possible relevance to the present application. No admission is necessarily intended, nor should be construed, that any of the preceding information constitutes prior art against the subject matter presented herein.

### SUMMARY

An aspect of the present application is to provide a composition for the management of HPV (Human Papillomavirus) infections.

In accordance with one aspect, there is provided a composition generally directed to a combination of active ingredients that may inhibit HPV viral replication likely through various mechanisms, thus providing a more comprehensive approach to managing such a viral infection.

In one aspect, the composition may be used for the management of HPV. In one embodiment, the composition comprises: activated folate, zinc (such as zinc sulfate or one or more other zinc compounds), a polyphenol catechin such as EGCG (epigallocatechin-3-gallate, which is typically found, for example, in green tea), broccoli seed or sprout, and one or more carotenoids. In some embodiments, the composition further comprises a mushroom or extract thereof, such as Ganoderma lucidum (“Reishi”) mushroom, Trametes versicolor (“Turkey tail”), Lentinula edodes (Shiitake mushroom), or any extract thereof.

The present composition may be useful in helping the immune system respond to HPV infections, likely reducing the frequency and severity of outbreaks.

Currently, preventative vaccines are available, but no specific antiviral treatment exists for HPV. Therefore, early-stage management primarily involves treating symptoms and screening for abnormal cell progression. Procedures exist for later stage cases which include cryotherapy, laser therapy, loop electrosurgical procedure, or cone biopsy to remove or destroy abnormal tissue. This highlights the urgent need for novel therapeutic approaches, such as the present formulation, that may be used to manage HPV infections effectively in early-stage cases.

The composition may be designed to target HPV through a multi-faceted approach, addressing both the virus and its potential complications. The formulation has surprisingly been found to be a combination of natural ingredients which may be acting in synergy and that may enhance the body's immune response, inhibit viral replication, and promote the elimination of infected cells. By doing so, the composition may reduce the severity and duration of HPV infections.

It is known that the individual components of the present composition may be effective in the management of HPV. However, the present composition provides a combination of components that may provide for a comprehensive approach to HPV management.

In one embodiment, a composition described herein comprises a combination of activated folate, zinc sulfate, EGCG, broccoli seed or sprout, and one or more carotenoids, for the management of HPV. Specifically, this combination may be more effective to any single individual component and any composition that does not contain one or more of these components. In one embodiment the composition comprises one or more zinc compounds, such as zinc sulfate, zinc gluconate, zinc acetate, zinc citrate, zinc picolinate, zinc oxide, zinc orotate, zinc aspartate, zinc methionine or zinc glycinate. In one embodiment, the zinc compound is zinc sulfate. For example, the inclusion of zinc sulfate may be effective, and in combination with the other components when compared to compositions that do not contain zinc, or other forms of zinc.

The composition may be used in response to low-grade changes, typically during the “watch and wait” period. Conventional medical care has patients “watch and wait” for a repeat future Pap smear test if the abnormal cell changes are considered low grade.

The composition may be made in a form for administration, and administered to a subject, in various forms, including but not limited to, oral capsules, tablets, liquid extracts, or topical creams, allowing for flexibility in delivery options.

### DETAILED DESCRIPTION

Certain vitamins, minerals and natural compounds have individually been shown to have a beneficial effect on HPV outbreak frequency and symptoms. For example, one benefit of activated folate is its bioavailability in the body. Zinc deficiency can lead to impaired immune responses. The positive effects of various fruits and vegetables, including sources of these compounds (such as broccoli seed/sprout, green tea, and carotenoids), are known. There is a need to address the negative effects of oxidative damage, inflammation, and compromised immunity, any or all of which are prevalent in HPV conditions.

Despite their individual roles in interfering with various cellular mechanisms typically associated with HPV infection, none have been shown to offer adequate management of HPV infections. Therefore, there is a need for a composition suitable for management of HPV infections, particularly given the various and complex mechanisms associated with HPV infection and replication, and its effects in those infected, including the impact of HPV on immunity, oxidative damage, and HPV-related cancers.

Provided herein is a composition that may be used in the management of HPV infections. As used herein, “management” (or “managing”) may include, for example, the control of symptoms associated with HPV infection in one or more individuals, and may also include reducing the frequency and/or persistence of HPV related conditions.

In general, the composition comprises a combination of activated folate, one or more zinc compounds, a polyphenol catechin, broccoli seed or sprout, and one or more carotenoids, for the management of HPV. In one embodiment, the zinc is zinc sulfate. Specifically, the composition comprises a combination which may act synergistically in managing HPV infection, as compared to any single individual component and any composition that does not contain one or more of these components.

In one embodiment, the composition comprises activated folate as 5-methyltetrahydrofolate (5MTHF), zinc as zinc sulfate, green tea extract containing EGCG, broccoli seed or sprout, Reishi mushroom (or extract), and mixed carotenoids.

In certain embodiments, the composition further comprises one or more mushrooms or mushroom extracts. In one embodiment, the mushroom is a Reishi mushroom or Reishi mushroom extract. In certain embodiments, the mushroom is Lentinula edodes (“Shiitake”) mushroom or extract thereof. In one embodiment, the Reishi mushroom extract is from a different form of a Reishi mushroom. In one embodiment, the mushroom is substituted with Trametes versicolor (“Turkey Tail”) mushroom or extract.

In one embodiment, the zinc is in the form of the compound zinc sulfate. In certain embodiments, the zinc is in the form of one or more other compound forms of zinc, such as zinc gluconate, zinc acetate, zinc citrate, zinc picolinate, zinc oxide, zinc orotate, zinc aspartate, zinc methionine or zinc glycinate.

In one embodiment, the polyphenol catechin is EGCG (epigallocatechin-3-gallate). In certain embodiments, the EGCG is derived from green tea extract. In certain embodiments, the EGCG is in the form of nut or berry that contains EGCG that is not from green tea leaf or green tea leaf extract.

In one embodiment, the folate is in the form of activated folate, such as 5-methyltetrahydrofolate (5MTHF). In certain embodiments, the folate is in the form of other forms of folate, such as L-Methylfolate, 10-Formyltetrahydrofolate or 5-Formyl Tetrahydrofolate.

Ideally, broccoli seed or sprout is used. However, in certain embodiments, the composition may comprise one or more broccoli metabolites. For example, the broccoli metabolites may include one or more of DIM (Diindolylmethane), I3C (Indole-3-carbinol), sulphoraphane, and glucosinolates. The broccoli metabolites may be from broccoli powder, such as broccoli sprout powder or broccoli seed powder. In some embodiments, the broccoli metabolites themselves may be used.

In one embodiment, the carotenoids are in the form of a fruit, nut, or algae extract containing a carotenoid, or are in the form of mixed carotenoids. In one embodiment, the carotenoids are in the form of non-mixed carotenoids. The carotenoids may be substituted with Vitamin A.

In one embodiment, the composition may further comprise one or more or all of Vitamin C, Vitamin E, Vitamin B12, lycopene Extract, selenium, and Astragalus polysaccharides from the Astragalus root. In one embodiment, the composition comprises Astragalus.

The composition may be administered to a subject in one or more different forms. In one embodiment, the composition may be in the form of a capsule for oral ingestion. The composition may also be in the form of, for example, a tablet, liquid, loose powder, cream, intravenous, or any other suitable form. In certain embodiments, the composition may comprise a lipid enclosure, enteric capsule, or delivery vehicle that improves bioavailability.

### EXAMPLES

To gain a better understanding of the compositions and methods described herein, the following examples are set forth. These examples, and any examples described herein, are for illustrative purposes only, and they should not limit the scope of the present application in any way.

### Example 1

A preliminary, open-label, observational pilot study to investigate the effects of the present composition on cervical changes associated with HPV.

SUBJECTS: 10 healthy females, aged 25 to 40 years old

### Inclusion Criteria:

1. Anatomically female subjects between 25 to 40 years of age.
2. History of repeatedly abnormal Pap smears (at least 2 abnormal Pap results within 2 years).
3. Initial abnormal Pap smear result, progression no further than CIN II.
4. Willingness to complete questionnaires associated with the study and to complete all clinic/laboratory visits.
5. Provide voluntary, written, informed consent to participate in the study.

### Exclusion Criteria:

1. Women with normal Pap smear results.
2. Abnormal Pap smear results beyond CIN II.
3. Women who are currently undergoing treatment for abnormal Pap smear results (i.e. cryotherapy, loop excision).
4. Women who are pregnant, breast feeding, or planning to become pregnant during the course of the trial.
5. Alcohol or drug abuse in past year.
6. Subject has a known allergy to the supplement's active or inactive ingredients.
7. Subjects with unstable medical conditions.
8. Individuals who are cognitively impaired and/or who are unable to give informed consent.
9. Individuals who have started a new supplement within 30 days.
10. Individuals who have started a new prescription medication <12 months before the start of the study.
11. Individuals who are taking medications for autoimmune conditions, including steroids and anti-inflammatories.
12. Individuals who are taking supplements which contain the same ingredients of the test product, or who have discontinued the use of these supplements <30 days before the start of the study.
13. Any other condition which, in the Investigator's opinion, may adversely affect the subject's ability to complete the study or its measures or which may pose significant risk to the subject.
14. Any other medication which, in the Investigator's opinion, may adversely affect the subject's ability to complete the study or its measures.

### Intervention:

Two capsules containing a formulation of the present composition per dose, administered BID to each subject. The formulation used per dose is provided in Table 1.

TABLE 1
Dosage Composition
Serving Size: 2 Capsules
Servings Per Container: 30

| | Amount | |
|---|---|---|
| | Per Serving | |
| Vitamin A (as Mixed Carotenoids) | 2400 | mcg RAE |
| Vitamin C (as Ascorbic Acid) | 90 | mg |
| Vitamin E (as D-Alpha Tocopheryl Acetate) | 40 | mg AT |
| Folate (as Quatrefolic ® | 800 | mcg DFE |
| (6S)-5-Methyltetrahydrofolic acid, glucosamine salt) | | |
| Vitamin B12 (as Methylcobalamin) | 2.4 | mcg |
| Zinc (as Zinc Sulfate) | 11 | mg |
| Selenium (as Selenomethionine) | 55 | mcg |
| Green Tea Leaf Extract (Decaffeinated) | 250 | mg |
| (80% Catechins, 50% EGCG, 98% Polyphenols) | | |
| Broccoli Sprout Powder (as HiActives ®) | 200 | mg |
| Astragalus (Astragalus membranaceus) Root Extract | 100 | mg |
| (5:1 extract, equivalent to 500 mg of fresh Astragalus) | | |
| Natural Lycopene Extract (containing 5% Lycopene) | 70 | mg |
| Reishi Mushroom Extract (30% Polysaccharides, | 50 | mg |
| equivalent to 500 mg of fresh Reishi) | | |

Daily Value not established.
Other Ingredients: Hypromellose (Capsule), Rice Flour, Magnesium Stearate

### Design:

An open-label, observational study to investigate the effects of a test product on improving the immune system response in healthy women who are HPV positive. At screening (visit 1—day <0), medical history and concomitant therapies were reviewed. Eligibility included a baseline questionnaire, Pap smear test, and initial bloodwork. At baseline (visit 2—day 0), eligible subjects proceeded to the laboratory where bloodwork was collected. Subjects then began supplementing 2 capsules twice daily of the test product. The subjects were required to receive another Pap smear test at week 16.

### Results & Discussion

### Exclusion and Drop Out Rates

When most participants were expected to receive bloodwork, the COVID-19 pandemic ensued, and many parts of Ontario were put into lockdown. This resulted in a normal study attrition rate of 20%.

One subject was excluded at baseline due to a normal Pap smear result. Another participant was excluded at baseline having recently undergone cryotherapy and a LEEP, resulting in a normal Pap result.

Eight participants completed the baseline and final Pap smear.

### HPV Vaccination:

3 of the 10 initial participants previously received an HPV vaccination.

### Bloodwork Results:

The initial bloodwork results were utilized as a baseline measure to assess for outright deficiencies commonly seen in women with HPV in the literature.

No significant findings were present in the bloodwork of participants who received baseline bloodwork assessments.

### Pap Smear Results:

Eight participants received final Pap smear results. Of the eight participants, five returned with results negative for intraepithelial lesions or malignancies, with no signs of abnormal cells. The most significant results were from two participants who initially had results of CIN II with years of previous ASCUS or abnormal Pap tests, reaching the end of the study resulting negative for intraepithelial lesions or malignancies. One of those two CIN II participants claimed abnormal Pap smear results since 2015, having her first negative Pap smear following the study. The final participant returned with atypical squamous cells of undetermined significance (ASCUS), originally testing for abnormal cells consistent with squamous intraepithelial lesions.

### CONCLUSION

Serum blood levels may not be a clinically significant measure of nutrient deficiencies, considering the complex relationship of storage and circulation of the nutrients in question. The study showed significantly beneficial results for HPV-related cervical changes in the participants when taking the composition. The composition has thus been shown to be a potentially effective intervention for women with HPV-related cervical changes to prevent further progression.

### EMBODIMENTS

The following are exemplary embodiments:

Embodiment 1: A composition for management of HPV infection comprising activated folate; one or more of zinc sulfate, zinc gluconate, zinc acetate, zinc citrate, zinc picolinate, zinc oxide, zinc orotate, zinc aspartate, zinc methionine, or zinc glycinate; a polyphenol catechin; broccoli seed or sprout; and one or more carotenoids.

Embodiment 2: The composition of Embodiment 1, further comprising one or more of a Ganoderma lucidum (Reishi) mushroom, a Trametes versicolor (Turkey Tail) mushroom, a Lentinula edodes (Shiitake) mushroom, or one or more extracts thereof.

Embodiment 3: The composition of Embodiment 1 or 2, wherein the activated folate is in the form of 5-methyltetrahydrofolate (5MTHF).

Embodiment 4: The composition of any of Embodiments 1 to 3, wherein the zinc is in the form of zinc sulfate.

Embodiment 5: The composition of any of Embodiments 1 to 4, wherein the polyphenol catechin is EGCG (epigallocatechin-3-gallate).

Embodiment 6: The composition of any of Embodiments 1 to 5, wherein the polyphenol catechin or EGCG is from Green Tea extract.

Embodiment 7: The composition of any of Embodiments 1 to 6, wherein the activated folate is in the form of other forms of folate, such as L-Methylfolate, 10-Formyltetrahydrofolate or 5-Formyl Tetrahydrofolate.

Embodiment 8: The composition of any of Embodiments 1 to 7, wherein the composition comprises one or more broccoli metabolites, such as Diindolylmethane, Indole-3-carbinol, Sulphoraphane or Glucosinolates.

Embodiment 9: The composition of Embodiment 8, wherein the broccoli metabolites are from broccoli sprout or seed powder.

Embodiment 10: The composition of any of Embodiments 1 to 9, wherein the zinc compound is one or more of zinc gluconate, zinc acetate, zinc citrate, zinc picolinate, zinc oxide, zinc orotate, zinc aspartate, zinc methionine, or zinc glycinate.

Embodiment 11: The composition of any of Embodiments 1 to 10, wherein the EGCG or catechin is in the form of nut or berry that contains EGCG that is not from Green Tea Leaf or Green Tea Leaf Extract.

Embodiment 12: The composition of Embodiment 9, wherein the broccoli metabolites are from Broccoli Sprout Powder, or wherein the broccoli sprout powder is substituted with Broccoli Powder, Broccoli Seed Powder, or just the Broccoli metabolites of DIM (Diindolylmethane), I3C (Indole-3-carbinol) Sulphoraphane or Glucosinolates are used.

Embodiment 13: The composition of any of Embodiments 2 to 12, wherein the mushroom or mushroom extract is Reishi mushroom or extract thereof.

Embodiment 14: The composition of any of Embodiments 2 to 13, wherein the Reishi mushroom or mushroom extract is in a different form of a Reishi mushroom.

Embodiment 15: The composition of any of Embodiments 2 to 14, wherein the mushroom or mushroom extract is Turkey Tail mushroom or extract thereof, or wherein the mushroom or mushroom extract is Shiitake mushroom or extract thereof.

Embodiment 16: The composition of any of Embodiments 1 to 15, wherein the carotenoids are in the form of a fruit, nut or algae extract containing a carotenoid, or are mixed carotenoids.

Embodiment 17: The composition of any of Embodiments 1 to 16, wherein the carotenoids are in the form of non-mixed carotenoids.

Embodiment 18: The composition of any of Embodiments 1 to 17, wherein the carotenoids are substituted with Vitamin A.

Embodiment 19: The composition of any of Embodiments 1 to 18, further comprising one or more or all of Vitamin C, Vitamin E, Vitamin B12, lycopene extract, selenium and Astragalus polysaccharides from the Astragalus root.

Embodiment 21: A composition comprising activated folate as 5-methyltetrahydrofolate (5MTHF), zinc as zinc sulfate, Green Tea Extract containing EGCG, broccoli seed or sprout, Reishi mushroom or extract thereof, and mixed carotenoids.

Embodiment 22: The composition of Embodiment 21, further comprising one or more of a Ganoderma lucidum (Reishi) mushroom, a Trametes versicolor (Turkey Tail) mushroom, a Lentinula edodes (Shiitake) mushroom, or any extract thereof.

Embodiment 23: The composition of any of Embodiments 1 to 22, in the form of a capsule for oral ingestion, or which is administered in capsule form orally to a subject.

Embodiment 24: The composition of any of Embodiments 1 to 23, in the form of a tablet.

Embodiment 25: The composition of any of Embodiments 1 to 24, in the form of a liquid.

Embodiment 26: The composition of any of Embodiments 1 to 25, in the form of a loose powder.

Embodiment 27: The composition of any of Embodiments 1 to 26, in the form of a cream.

Embodiment 28: The composition of any of Embodiments 1 to 27, in an intravenous form.

Embodiment 29: The composition of any of Embodiments 1 to 28, further comprising a lipid enclosure, enteric capsule, or delivery vehicle that improves bioavailability.

Embodiment 30: A method of managing an HPV infection in a subject comprising administering the composition of any of Embodiments 1 to 29 to the subject.

The above disclosure is intended to be illustrative and not exhaustive. The description will suggest many variations and alternatives to one of ordinary skill in the art. Those familiar with the art may recognize other equivalents to the specific embodiments described herein within, without departing from the spirit and scope thereof.

What is claimed is:

1. A composition for management of an HPV infection comprising:
activated folate,
a zinc compound which is one or more of zinc sulfate, zinc gluconate, zinc acetate, zinc citrate, zinc picolinate, zinc oxide, zinc orotate, zinc aspartate, zinc methionine, or zinc glycinate sulfate,
a polyphenol catechin,
broccoli seed or sprout, and
one or more carotenoids.

2. The composition of claim 1, wherein the zinc compound is zinc sulfate.

3. The composition of claim 1, further comprising one or more of a Ganoderma lucidum (Reishi) mushroom, a Trametes versicolor (Turkey Tail) mushroom, a Lentinula edodes (Shiitake) mushroom, or one or more extracts thereof.

4. The composition of claim 1, wherein the activated folate is in the form of 5-methyltetrahydrofolate (5MTHF).

5. The composition of claim 1, wherein the polyphenol catechin is EGCG (epigallocatechin-3-gallate).

6. The composition of claim 1, wherein the polyphenol catechin is derived from or in the form of a green tea extract.

7. The composition of claim 1, wherein the activated folate is in the form of L-methylfolate, 10-formyltetrahydrofolate or 5-formyl tetrahydrofolate.

8. The composition of claim 1, wherein the composition further comprises one or more broccoli metabolites which are one or more of diindolylmethane, indole-3-carbinol, sulphoraphane, or glucosinolates.

9. The composition of claim 1, wherein the catechin is in the form of a nut or berry that contains EGCG that is not from green tea leaf or green tea leaf extract.

10. The composition of claim 8, wherein the one or more broccoli metabolites are from broccoli sprout powder, or wherein the broccoli sprout powder is substituted with broccoli powder, broccoli seed powder, or broccoli metabolites of DIM (Diindolylmethane), I3C (Indole-3-carbinol) sulphoraphane, or glucosinolates.

11. The composition of claim 1, wherein the one or more carotenoids are one or more of α-carotene, β-carotene, β-cryptoxanthin, lutein, zeaxanthin, astaxanthin, or lycopene.

12. The composition of claim 1, wherein the one or more carotenoids are in the form of a fruit, nut, or algae extract containing carotenoids.

13. The composition of claim 1, wherein the one or more carotenoids are substituted with Vitamin A.

14. The composition of claim 1, further comprising one or more of Vitamin C, Vitamin E, Vitamin B12, lycopene extract, selenium, or Astragalus polysaccharides from the Astragalus root.

15. The composition of claim 1, in the form of a capsule, tablet, liquid, or loose powder for oral ingestion, in an intravenous form, or in a suppository form.

16. The composition of claim 15, further comprising a lipid enclosure, enteric capsule or delivery vehicle that improves bioavailability.

17. A composition comprising:
activated folate as 5-methyltetrahydrofolate (5MTHF),
zinc sulfate,
green tea extract containing EGCG,
broccoli seed or sprout,
Reishi mushroom or an extract thereof, and
one or more carotenoids.

18. The composition of claim 17, further comprising Astragalus.

19. The composition of claim 17, in the form of a capsule, tablet, liquid, or loose powder for oral ingestion, in an intravenous form, or in a suppository form, and optionally comprising a lipid enclosure, enteric capsule or delivery vehicle that improves bioavailability.

20. A method of managing an HPV infection in a subject comprising administering to the subject the composition of claim 1.
